(19) United States
(12) Patent Application Publication
(10) Pub. No.: US 2026/0276668 A1
(43) Pub. Date: Sep. 17, 2026

(54) AUTOMATED CELL PASSAGING

(71) Applicants: Markus BRÜDERL, Puch Bei Hallein (AT); Emilie KEIDEL, Puch Bei Hallein (AT); Molecular Devices (Austria) GmbH, Puch Bei Hallein (AT); Molecular Devices, LLC, San Jose, CA (US)

(72) Inventors: Markus Brüderl, Puch Bei Hallein (AT); Emilie Keidel, Puch Bei Hallein (AT); Zhisong Tong, San Jose, CA (US); Angeline Lim, Scotts Valley, CA (US); Prathyushakrishna Macha, San Jose, CA (US)

(21) Appl. No.: 19/165,067

(22) PCT Filed: Mar. 16, 2024
(86) PCT No.: PCT/US2024/020330

§ 371 Date: Sep. 12, 2025

Related U.S. Application Data
(60) Provisional application No. 63/511,930, filed on Jul. 5, 2023.

(51) Int. Cl. G01N 35/10; C12M 1/26; C12M 1/32; C12M 1/36

(52) U.S. Cl. CPC G01N 35/1011; C12M 23/12; C12M 33/04; C12M 41/48; G01N 2035/1048

(57) ABSTRACT

Disclosed are methods of identifying the location of the of an extracellular matrix structure on the surface of a culture plate with a substantially automated process. Also disclosed are automated methods for removal of cells from an extracellular matrix structure within which the cells are grown. The method includes providing cultured cells in the extracellular matrix structure on a surface of a culture plate; and using a substantially automated process, disrupting the extracellular matrix structure at an X, Y position of the extracellular matrix structure on the surface of the culture plate with a pipette tip associated with a liquid handler.

### CROSS-REFERENCE TO RELATED APPLICATIONS

This application is the national phase of PCT International application No. PCT/US2024/020330 filed on Mar. 16, 2024, which claims the benefit of and priority to PCT International application No. PCT/US2023/064645, filed Mar. 17, 2023, and U.S. Provisional Application No. 63/511,930, filed, Jul. 5, 2023 the entire contents of each of which are incorporated herein by reference.

### TECHNICAL FIELD

This disclosure relates to the culture of cells. More specifically this disclosure relates to the use of procedures to passage and culture cells such as organoids.

### BACKGROUND

Culturing cells in a three-dimensional (3D) environment yields cellular behavior and morphology that more closely matches what is observed in the human body. 3D hydrogels/hydroscaffolds used for this kind of culturing have a unique attribute: cells can be deposited in specific locations in 3D space and remain in position for extended time periods. This enables the creation of structures (e.g., spheroids, tumoroids, organoids, and/or other multi-cellular bodies) and co-culture environments where cellular interactions and developments over time are observed.

Organoids are three-dimensional miniature models of organs that are grown in vitro (in a laboratory setting) from stem cells or tissue samples. They mimic the cellular or tissue structure and function of real organs and offer a unique platform for studying human biology and disease. Organoids should not be thought of as organ replicas. Organoids are not vascularized, that is they are missing blood vessels and other system components.

Organoids are typically derived from pluripotent stem cells (such as embryonic stem cells or induced pluripotent stem cells) or adult stem cells (such as intestinal stem cells). These cells are induced to differentiate into specific cell types and self-assemble into complex structures resembling an organ.

Organoids are composed of multiple cell types and display organized tissue architecture, including distinct cell layers, similar to the organ they are modeling. They may contain functional units, such as blood vessels, glands, or neuronal networks, depending on the organ being studied.

### SUMMARY

In a first aspect the disclosure provides a substantially automated process for the identification of the position of an extracellular matrix (ECM) structure on the surface of a culture plate.

In a second aspect, the disclosure provides an automated method for removal of cells from an extracellular matrix structure within which the cells are grown on a surface of a culture plate. A substantially automated process is used to disrupt the extracellular matrix structure at an X, Y position of the extracellular matrix structure on the surface of the culture plate with a pipette tip associated with a liquid handler.

In a third aspect, the disclosure provides a method for fragmentation of organoids. The method includes centrifuging a cell solution in a container to form a cell pellet and a supernatant; aspirating a first volume of the supernatant at a first fixed height from the bottom of the container and a first fixed speed; and aspirating a second volume of the supernatant at a second fixed height from the bottom of the container and a second fixed speed.

In a fourth aspect, the disclosure provides a method for fragmentation of organoids. The method includes, in a container comprising an organoid pellet and a supernatant of a first volume, a) aspirating and dispensing a second volume at a first fixed height from the bottom of the container multiple times sequentially; b) aspirating a third volume at a second fixed height from the bottom of the container and dispensing the third volume back into the container; and c) repeating the steps a) and b).

In a fifth aspect, the disclosure provides a method for seeding of a solution of cells. The method includes a) mixing, via aspirating and dispensing, about 60% of the volume of the solution of cells multiple times; b) aspirating a portion of the mixed solution to provide an aspirated solution and remaining non-aspirated solution; c) dispensing at least a portion of the aspirated solution to a culture plate not previously used to culture the cells; and d) repeating steps a) through c) with the remaining non-aspirated solution.

Further aspects and embodiments are provided in the foregoing drawings, detailed description and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 is a schematic representation of one embodiment of a substantially automated process for determining the x,y coordinates of various ECM structures

FIG. 2 is an example of an image of a cell culture surface being used to create image masks.

FIG. 3 is an example of a set of image masks being used to generate a series of x,y, coordinates that define the locations of ECM structures.

FIG. 4 is a representation of transferring the x,y coordinates of an ECM structure to a liquid handler.

FIG. 5 is a schematic representation of one embodiment of a substantially automated process for the creation of an image mask.

FIG. 6 is an example of contrast inversion for an image of a culture plate.

FIG. 7 is an example of image blurring for an image of a culture plate.

FIG. 8 is an example of mask creation for an image of a culture plate.

FIG. 9 is an example of combination of masks of the individual cells and/or cell clusters to generate a mask for the entire ECM structure for an image of a culture plate.

FIG. 10 depicts micrographs showing comparison of the well before and after passaging with liquid handler. Most of the organoids have been recovered. What is left in are the single layer cells that are not as dense as organoids and sticky to the bottom of the well.

FIG. 11 shows exemplary pipettor movement patterns of predetermined positions calculated for hydrogel dome breaking using preceding formulae with 6 steps (upper left panel), 7 steps (upper right panel), 8 steps (second row left panel), and 9 steps (second row right panel). When using theta value of 15.7, a spiral pattern of steps is formed for each pattern. Also depicted are exemplary pipettor movement patterns of predetermined positions calculated for hydrogel dome breaking using preceding formulae with 10 steps (third row left panel), 11 steps (third row right panel), 12 steps (lower left panel), and 13 steps (lower right panel). When using theta value of 15.7, a spiral pattern of steps is formed for each pattern.

FIG. 12 shows exemplary pipettor movement patterns calculated for hydrogel dome breaking using preceding formulae with wherein diameter (mm)=8, number of steps=13, alpha=diameter/1.8, and theta=30. When using theta value of 30, the pattern of steps appears less like a spiral and a star-like pattern begins to emerge.

FIG. 13 is a depiction of the 17-point breaking positions.

FIG. 14 is a 96 deep well plate (left) and the adapter for cooling (right).

FIG. 15 is micrographs showing the growth of the organoids that are passaged and seeded via liquid handler at (A) Day 1, (B) Day 2, (C) Day 5 and (D) Day 7.

FIG. 16 is a table showing the composition needed to make 50% Matrigel.

FIG. 17 is a table showing the composition needed to make 80% Matrigel.

FIG. 18 is an example of the mask generated from IN Carta to detect the organoids from the background and measure the area of the masks.

FIG. 19 is a table detailing the area corresponding to FIG. 18.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

The term “and/or” refers to and encompasses any and all possible combinations of one or more of the associated listed items.

The term “about,” when referring to a measurable value such as an amount of a compound, dose, time, temperature, and the like, is meant to encompass variations of 10%, 5%, 1%, 0.5%, or even 0.1% of the specified amount.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

The terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this disclosure belongs. In the event of conflicting terminology, the present specification is controlling.

“Substantially automated” such as in a “substantially automated process” as used herein refers to a process, system, or task where machines or algorithms handle the majority of the work, with minimal human intervention required for its core function. Human oversight or input may still be necessary, but it is limited to exceptional circumstances, specific decision points, or for quality control.

All patents, patent applications and publications referred to herein are incorporated by reference in their entirety.

The embodiments described in one aspect of the present disclosure are not limited to the aspect described. The embodiments may also be applied to a different aspect of the disclosure as long as the embodiments do not prevent these aspects of the disclosure from operating for its intended purpose.

Organoid, spheroid, tumoroid, and three-dimensional (3D) cell culture models are useful in many applications such as disease modeling and regenerative medicine. 3D cellular models like organoids and spheroids may be useful to better understand complex biology in a physiologically relevant context because cells often retain natural shape and proper spatial orientation, such as in aggregates or spheroids, whereas 2D models of cells grown in a sheet or monolayer may not be as successful. Gene and protein expression of 3D cell culture may more closely mimic gene and protein expression. For example, 3D cell cultures may be useful for drug target identification, lead compound identification, compound optimization, preclinical attesting, solid tumor modeling, genetic disease modeling, drug discovery, precision medicine, organs-on-chips, and bioprinting.

The term “spheroids” refer to three dimensional (3D) multicellular in vitro tissue cultures aggregates composed of one or more cell types that grow and proliferate, and may exhibit enhance physiological responses, but do not undergo differentiation or self-organization. Common cell sources for spheroids are primary tissues or immortalized cell lines. Spheroids may bridge the gap between monolayers and complex organs.

The term “tumoroid” refers to three dimensional (3D) multicellular in vitro tissue culture aggregates composed of one or more cell types typically derived from primary tumors harvested from oncological patients and can mimic human tumor microenvironment. Tumoroids may be useful for studies on novel cancer drugs or for use in precision medicine in the field of oncology. Cancer cell lines may be, for example, bladder, breast, colon, hematopoietic and lymphoid, liver, lung, ovary, prostate, skin, and the like.

The term “stem cells” refers to undifferentiated cells that have the potential to develop into many different cell types that carry out different functions. Pluripotent stem cells, such as those found in embryos, can give rise to any type of cell such as those in brain, bone, heart, and skin. Some human adult cells can be reprogrammed into embryonic stem cell-like state called induced pluripotent stem cells (iPSCs). Multipotent stem cells, for example, found in adults or in babies' umbilical cords, may develop into the cells that make up the organ system that they originated from. When grown under certain cell culture conditions, pluripotent stem cells can remain undifferentiated. To generate differentiated cells, the chemical composition of the culture medium may be changed, the surface of the culture dish may be altered, or the cells may be modified by forcing expression of certain genes.

As used herein, “Organoids” is meant to refer to self-organizing structures that exhibit multiple cell types and recapitulate the architecture and function of specific organs or tissues. Organoids are three dimensional (3D) multicellular in vitro tissue culture aggregates composed of one or more cell types, in which cells spontaneously self-organize into properly differentiated functional cell types and progenitors that resemble their in vivo counterparts in at least one aspect. Organoids mimic their corresponding in vivo organs. Organoids can be derived from pluripotent stem cells (PSCs), induced pluripotent stem cells (iPSCs), neonatal tissue stem cells, embryonic stem cells (ESCs), adult stem cells, or primary tissue. Organoid cultures can be crafted to resemble much of the complexity of an organ, therefore are useful for study of disease etiology and treatment. Organoid technology has recently emerged as an essential tool for both fundamental and biomedical research. The organoid cultures may be selected from different types of target organs such as, e.g., lung, intestine such as small intestine, colon, stomach, pancreas, liver, kidney, skin, bone marrow, blood-brain barrier, brain, heart, and the like.

Development: Organoids are typically derived from pluripotent stem cells (such as embryonic stem cells or induced pluripotent stem cells) or adult stem cells (such as intestinal stem cells). These cells are induced to differentiate into specific cell types and self-assemble into complex structures resembling an organ.

Structure and complexity: Organoids are composed of multiple cell types and display organized tissue architecture, including distinct cell layers, similar to the organ they are modeling. They may contain functional units, such as blood vessels, glands, or neuronal networks, depending on the organ being studied.

Disease modeling: Organoids are regularly used in disease research by providing a more accurate representation of human physiology compared to traditional cell cultures or animal models. Researchers may generate organoids from patient-derived cells, allowing them to study diseases in a personalized manner and investigate the underlying mechanisms of diseases, drug responses, and potential treatment strategies.

Drug discovery and testing: Organoids have significant implications for drug development. They may be used to test the safety and efficacy of potential drugs before clinical trials, reducing reliance on animal testing and improving the success rate of clinical trials. Organoids also enable the study of drug responses in specific patient populations, leading to personalized medicine approaches.

Regenerative medicine: Organoids hold promise for regenerative medicine and tissue engineering. By using patient-derived stem cells, scientists aim to generate functional organoids that could be transplanted into individuals with organ damage or failure, serving as personalized and functional replacement tissues or organs.

Organoids do not fully replicate the complexity of whole organs, and they lack interactions with the body's circulatory, immune, and nervous systems. Additionally, scaling up the production of organoids to a clinically relevant scale remains a challenge.

Organoid types: There are numerous types of organoids developed to study different organs, including brain organoids (cerebral organoids), liver organoids, kidney organoids, intestinal organoids, lung organoids, and more.

Organoid research is a rapidly evolving field with vast potential for advancing our understanding of human biology, disease mechanisms, and therapeutic development. It offers a bridge between simplistic in vitro models and in vivo studies, contributing to advancements in personalized medicine and regenerative therapies.

Organoids are propagated through a series of steps that involve the initial establishment of organoid cultures and subsequent passaging or subculturing to maintain and expand the organoid population. The specific propagation methods may vary depending on the organoid type and the research protocols used. However, here is a general overview of the process:

Establishment of organoid cultures: Isolation and preparation of stem cells or tissue: Organoids may be derived from pluripotent stem cells (PSCs) or adult stem cells, depending on the organ being studied. PSCs may be obtained from embryonic stem cells (ESCs) or induced pluripotent stem cells (iPSCs), while adult stem cells are often isolated from specific tissues. Organoids may also be obtained from existing organoids, for example by the fragmentation of existing organoids.

For example, stomach organoid tissue cultures may be derived from a source such as, e.g., adult mouse, adult human, hPSC, and the like. Stomach organoid tissue cultures may employ a stem cell culture condition (niche factors) including media components such as one or more of EGF, Noggin, R-spondin, Wnt-3A, FGF10, and the like, depending on source. The differentiation culture condition may include EGF, R-spondin EGF, R-spondin, and the like, depending on source.

As another example, small intestine organoid tissue cultures may be derived from a source such as, e.g., adult mouse, adult human, hPSC, and the like. Small intestine organoid tissue cultures may employ a stem cell culture condition (niche factors) including media components such as one or more of EGF, Noggin, R-spondin, Wnt-3A TGF-beta inhibitor, p38 inhibitor, and the like, depending on source. The differentiation culture condition may include EGF, Noggin, TGF-beta inhibitor and the like, depending on source.

As a further example, colon organoid tissue cultures may be derived from a source such as, e.g., adult mouse, adult human, and the like. Colon organoid tissue cultures may employ a stem cell culture condition (niche factors) including media components such as one or more of EGF, Noggin, R-spondin, Wnt-3A TGF-beta inhibitor, p38 inhibitor, and the like, depending on source. The differentiation culture condition may include EGF, Noggin, TGF-beta inhibitor and the like, depending on source.

As another example, pancreas organoid tissue cultures may be derived from a source such as, e.g., adult mouse, adult human, and the like. Pancreas organoid tissue cultures may employ a stem cell culture condition (niche factors) including media components such as one or more of EGF, Noggin, R-spondin, Wnt-3A, FGF10, nicotinamide, and the like, depending on source. The differentiation culture condition may include EGF, Noggin, R-spondin, Wnt-3A, and the like, depending on source.

As a further example, liver organoid tissue cultures may be derived from a source such as, e.g., adult mouse, adult human, and the like. Liver organoid tissue cultures may employ a stem cell culture condition (niche factors) including media components such as one or more of EGF, Noggin, R-spondin, Wnt-3A, FGF10, HGF, nicotinamide, and the like, depending on source. The differentiation culture condition may include EGF, Noggin, R-spondin, Wnt-3A, FGF10, TGF-beta inhibitor, Notch inhibition, BMP7, and the like, depending on source.

Differentiation and encapsulation: The stem cells may be induced to differentiate into the desired cell type(s) using specific growth factors and culture conditions. The differentiated cells may then be embedded or encapsulated in a supportive extracellular matrix (ECM), such as Matrigel or other hydrogels, that mimics the organ's native microenvironment.

Culture medium: Organoids require specialized culture media that provide the necessary nutrients, growth factors, and signaling molecules for their growth and development. The culture medium is usually supplemented with factors that promote organ-specific differentiation and expansion. Different media components may be required for each type of source cells used, and the type of differentiation to be achieved. Growth factors such as EGF, Noggin (NOG), R-spondin (RSPO1), HGF, BMP, FGF, and the like may be essential components of organoid media. The tissue culture media may comprise growth factors. The growth factors may be generated by the feeder cells. The growth factors may be recombinant growth factors. The recombinant growth factor proteins for organoid culture may include, for example, recombinant human EGF protein, recombinant HGF proteins such as, for example, human HGF protein, cynomolgus HGF protein, human FGF10, human Noggin/NOG protein, human RSPO1 protein, human BMP-2 protein, and the like. Additional recombinant growth factors for organoid culture may include, for example, EGF, FGF2, FGF7, FGF9, FGF10, HGF, NOG, RSPO1, RSPO3, Activin A, BMP2, and BMP4, and the like. The tissue culture media, or recombinant growth factor proteins for organoid culture, may be commercially available from, for example, Sino Biological, Inc., or Thermo Fisher Scientific.

3D cellular models like organoids and spheroids may be cultivated in a tissue culture media comprising a hydrogel, such as in a hydrogel dome within the media.

The term “hydrogel” or “hydrogels” refers to an extracellular matrix useful for culturing organoids. The hydrogel may include murine EHS sarcoma matrix, for example, available commercially as Matrigel (Corning), Cultex (Trevigen), Geltrex (Gibco), collagen type I, fibrin, hyaluronic acid (HA), gelatin methacrylate (GelMA), decellularized matrices, or biopolymers such as alginate, silk, nanocellulose; engineered materials such as polyethylene glycol (PEG), self-assembling peptides such as RADA16/PuraMatrix bQ13, poly (lactic/(co) glycolic) acid, polycaprolactone, polyacrylamide, oligo (ethylene glycol)-substituted polyisocyanopeptides, ELP (elastin-like protein), or combinations of these polymers.

Passaging or subculturing of organoids: Dissociation of organoids: To passage organoids, they may be first enzymatically or mechanically dissociated into smaller clusters or single cells. This step aims to break down the organoid structure and disperse the cells for further culturing.

Re-embedding or replating: The dissociated organoid cells may then be re-embedded or replated in fresh ECM and/or replated in a new culture vessel, depending on the specific protocols. Re-embedding involves mixing the cells with fresh ECM and allowing them to self-assemble into new organoids. Replating may involve transferring the cells onto a new culture dish or plate.

One example of an ECM is a hydrogel. The term “hydrogel” or “hydrogels” refers to an extracellular matrix useful for culturing organoids. The hydrogel may include murine EHS sarcoma matrix, for example, available commercially as Matrigel (Corning), Cultex (Trevigen), Geltrex (Gibco), collagen type I, fibrin, hyaluronic acid (HA), gelatin methacrylate (GelMA), decellularized matrices, or biopolymers such as alginate, silk, nanocellulose; engineered materials such as polyethylene glycol (PEG), self-assembling peptides such as RADA16/PuraMatrix bQ13, poly (lactic/(co) glycolic) acid, polycaprolactone, polyacrylamide, oligo (ethylene glycol)-substituted polyisocyanopeptides, ELP (elastin-like protein), or combinations of these polymers.

Expansion and differentiation: After passaging, the organoid cells are cultured in fresh medium to allow for growth and maturation. The culture conditions may be adjusted to promote specific differentiation pathways or maintain the desired cell types.

### Maintenance and Long-Term Culture:

Regular feeding and medium changes: cells need regular feeding and medium changes to maintain optimal conditions for growth and survival. This typically involves replacing a portion of the culture medium with fresh medium containing the necessary nutrients and growth factors.
Monitoring and quality control: cells should be regularly monitored for their growth, structural integrity, and cell type composition. Quality control measures, such as genetic or protein analysis, may be employed to ensure the organoids retain their desired characteristics and functionality.

It is important to note that the propagation methods may differ depending on the specific research protocols, organoid type, and desired applications. Optimization and customization of the protocols are often performed to achieve the best results for each specific organoid model.

Typically, cell passaging is performed manually. This is often time consuming. Additionally, manual passaging may be inconsistent and less precise due to individual tendencies and care. Even so, there are general protocols for passaging. The major steps of the manual process for passaging cells growing in an ECM includes breaking the ECM (Corning® Matrigel®), dissociating/fragmenting the cells, seeding the cells.

Due in part to the time-consuming nature of manual passaging cells grown in an ECM, described herein are methods for passaging of cells. The major steps of the process include breaking an ECM (e.g. Corning® Matrigel®) structure (such as a dome) containing the cells, dissociating/fragmenting the cells, seeding the cells.

### Identification of ECM Location

Described herein are methods for the removal of cells from an extracellular matrix structure. The method includes providing cultured cells in the extracellular matrix structure on a surface of a culture plate; identifying the location of the extracellular structure on the surface of the culture plate; and breaking the extracellular matrix structure by puncturing or slashing using a substantially automated process. The methods described herein may be used in any combination with the other method described herein, including but not limited to, methods of removing cells from ECM, methods of removing a supernatant from a cell pellet, methods of fragmenting cell clusters, and methods of seeding a solution of cells.

During cell culture in an ECM, a cell culture surface may hold one or more ECM structures in which the cells are being grown. One example of an ECM structure is a dome. The methods and systems described herein may include, prior to breaking an ECM structure, identifying the locations of ECM structures. In embodiments, the identification of ECM structure locations may be performed primarily by a human or using a substantially automated process.

Where a human is locating the ECM structures on the surface of the surface of a cell culture plate, the locations may be noted in x,y coordinates corresponding to locations on the surface. The x,y coordinates are recorded in a format such that they are machine readable/retrievable. By way of nonlimiting example, a human, via visual inspection (e.g. utilizing a microscope or other device), may identify the location of various ECM structures on a culture plate. The human may then record or cause to be recorded the x,y location of an ECM structure. Examples of human recordation include writing or entering the location in a storage device of any kind. Where the human causes the x.y location to be recorded, this may be through the push of a button or other signal that results in the automatic recordation of the x.y location determined by visual inspection.

Where locating the ECM structures is performed using a substantially automated process, the locations may be noted in x,y coordinates corresponding to locations on the surface. The x,y coordinates are recorded in a format such that they are machine readable/retrievable. Examples of locating by using a substantially automated process include, but are not limited to, detection methods using one or more sensors. For example, the x,y coordinates of various ECM structures may be detected using optical, electromagnetic, or physical/mechanical sensors. In the case of optical sensors, the x,y coordinates of various ECM structures may be located by differentiating from background using standard image processing or artificial intelligence/machine learning techniques. In certain embodiments, an image of the cell culture surface is captured and used to identify the x,y coordinates of various ECM structures. Where physical physical/mechanical sensors are used, the x,y coordinates of various ECM structures may be located by deforming or impeding a probe moving across or near the surface of the culture plate. In further embodiments, the x,y coordinates of various ECM structures may be identified by the use of electromagnetic sensors. Examples of such sensors include, but are not limited to, laser, lidar, radar, infrared, light diffraction, and/or stimulation/emission-based sensors.

In some embodiments, the x,y coordinates of various ECM structures may be identified by the location of any cells. Where cells are primarily located and/or clustered within an ECM structure, the identification of cells and/or clusters of cells provides the location of ECM structures. In embodiments, the identification of cell locations can be by any method including detected using optical, electromagnetic, or physical/mechanical sensors as set forth for the direct detection of ECM structures.

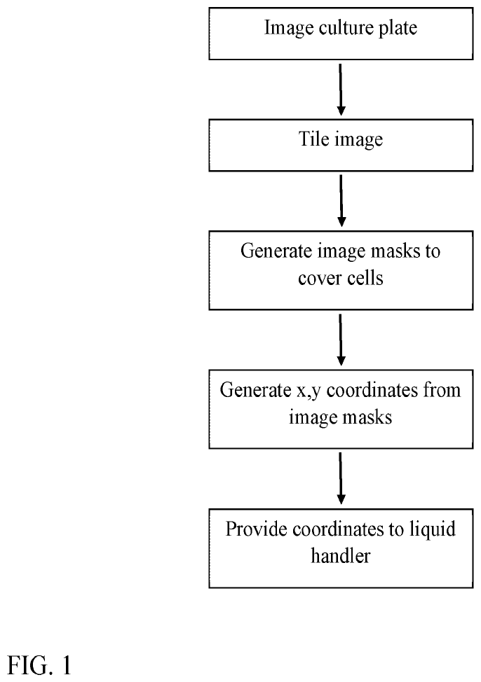

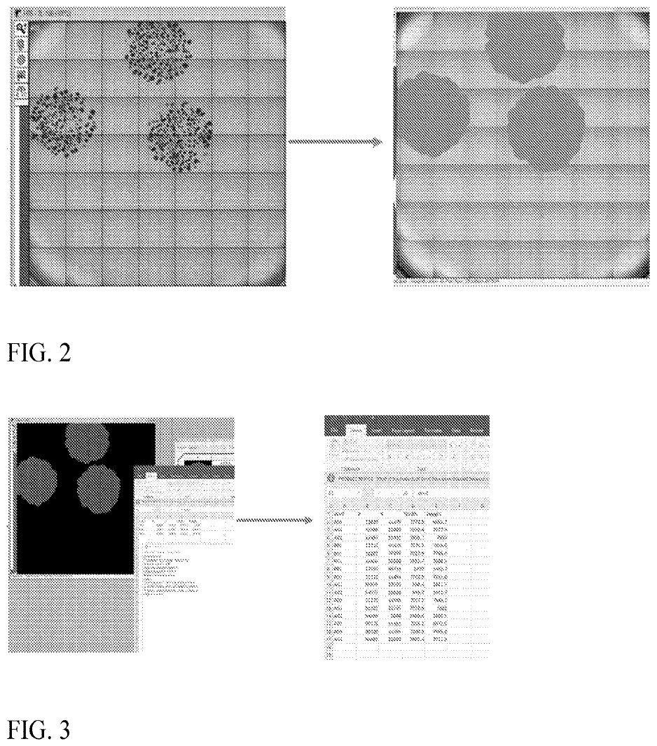

FIG. 1 provides a schematic representation of one embodiment of a substantially automated process for determining the x,y coordinates of various ECM structures. The first step provided therein is imaging the culture plate. That image may be tiled if required for masking or for the placements of coordinate grids. The tiled image may be used to create a mask to cover the cells and thus identify the locations of ECM structures. FIG. 2 provides one example of an image of a cell culture surface being used to create image masks.

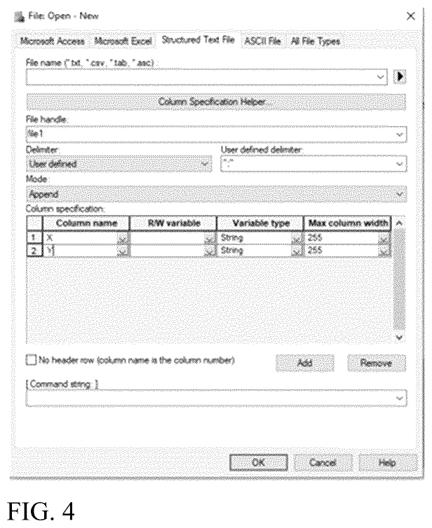

Once the masks are created, x,y coordinates of the masks may be generated. FIG. 3 provides one example of a set of image masks being used to generate a series of x,y, coordinates that define the locations of ECM structures. Once the x,y, coordinates of ECM structures have been determined, optionally, the locations may be provided to another device, such as liquid handler. The transfer of coordinates to a liquid handler is depicted in FIG. 4.

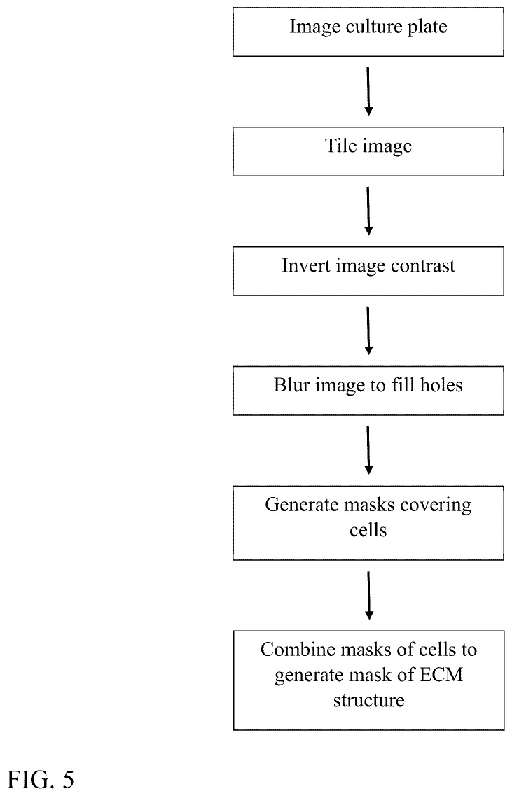

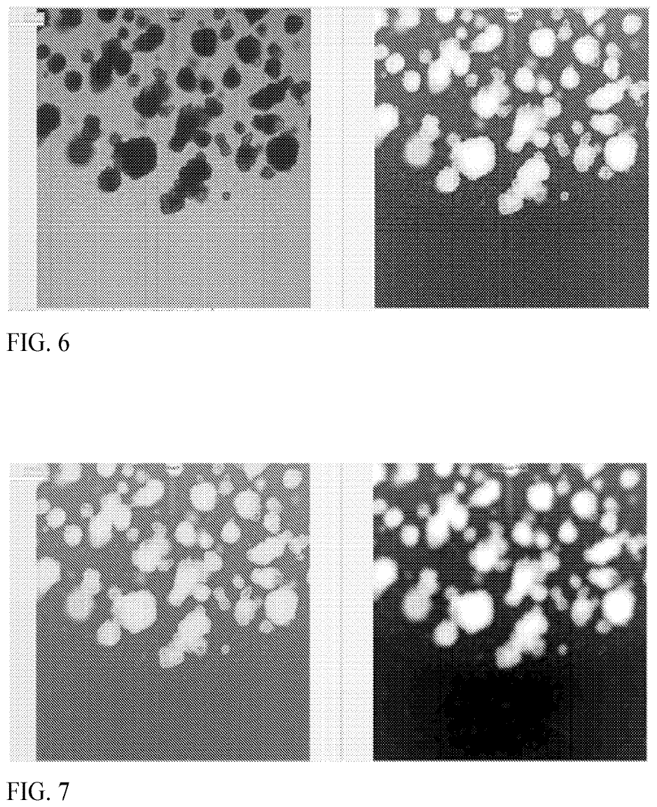

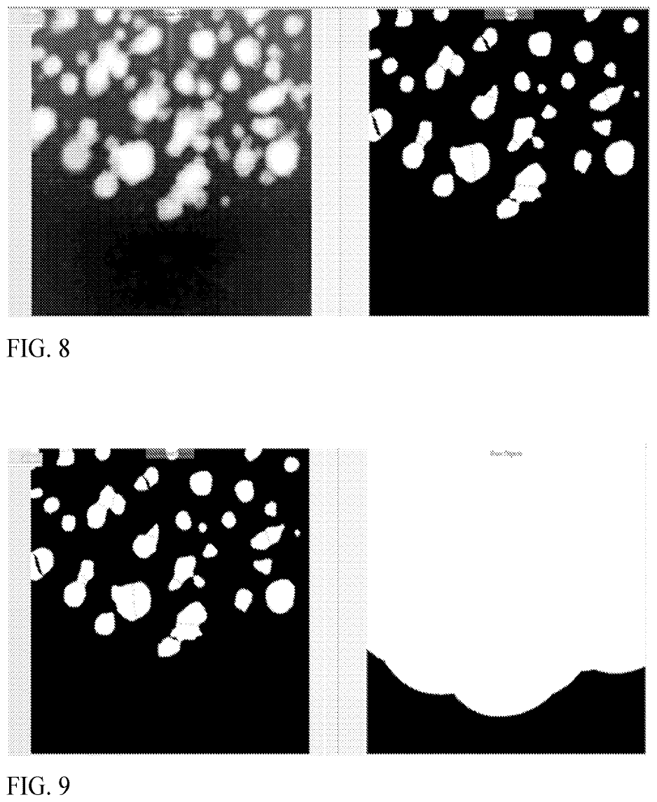

One embodiment for the creation of an image mask for the ECM structures is schematically represented in FIG. 5. In this embodiment, after imaging the culture plate and tiling the image, the contrast of the image is inverted. An example of such contrast inversion for an image of a culture plate is provided in FIG. 6. Once the inverted contrast image is obtained, the image may be blurred to fill in holes between and among cells in the image. An example of such image blurring is provided in FIG. 7. Masks may then be generated from the blurred image. An example of such mask creation from an image of cells is provided in FIG. 8. The masks of the individual cells and/or cell clusters may then be combined to generate a mask for the entire ECM structure. An example of the combination of masks of the individual cells and/or cell clusters to generate a mask for the entire ECM structure is provided in FIG. 9.

As will be appreciated, any combination of the forgoing steps is contemplated herein. Where the six steps set forth in FIG. 5 are, in order, steps A-F: a particular embodiment includes steps A, B, and F. Where the six steps set forth in FIG. 5 are, in order, steps A-F: a particular embodiment includes steps A, C, and F. Where the six steps set forth in FIG. 5 are, in order, steps A-F: a particular embodiment includes steps A, D, and F. Where the six steps set forth in FIG. 5 are, in order, steps A-F: a particular embodiment includes steps A, E, and F. Where the six steps set forth in FIG. 5 are, in order, steps A-F: a particular embodiment includes steps A, B, C, and F. Where the six steps set forth in FIG. 5 are, in order, steps A-F: a particular embodiment includes steps A, B, D, and F. Where the six steps set forth in FIG. 5 are, in order, steps A-F: a particular embodiment includes steps A, B, E, and F. Where the six steps set forth in FIG. 5 are, in order, steps A-F: a particular embodiment includes steps A, C, D, and F. Where the six steps set forth in FIG. 5 are, in order, steps A-F: a particular embodiment includes steps A, C, E, and F. Where the six steps set forth in FIG. 5 are, in order, steps A-F: a particular embodiment includes steps A, D, E, and F. Where the six steps set forth in FIG. 5 are, in order, steps A-F: a particular embodiment includes steps A, B, C, D, and F. Where the six steps set forth in FIG. 5 are, in order, steps A-F: a particular embodiment includes steps A, B, C, E, and F. Where the six steps set forth in FIG. 5 are, in order, steps A-F: a particular embodiment includes steps A, B, D, E, and F. Where the six steps set forth in FIG. 5 are, in order, steps A-F: a particular embodiment includes steps A, C, D, E, and F. Where the six steps set forth in FIG. 5 are, in order, steps A-F: a particular embodiment includes steps A, B, C, D, E, and F.

Removal of Cells from ECM.

Described herein are substantially automated processes for removal of cells from an ECM structure. In one embodiment, a liquid handler is provided the x,y coordinates of an ECM structure on a cell culture plate. In some aspects, the x,y coordinates of an ECM structure may be obtained via a substantially automated process as described herein or may be provided through a non-automated process. The liquid handler uses a pipette tip associated therewith to disrupt the ECM structure at the x,y coordinates and thus free cells from within the ECM structure. By way of example, the pipette tip may disrupt the ECM structure in any matter including, but not limited to piercing the ECM structure, breaking the ECM structure, or by slashing and/or dragging the pipette tip through the ECM structure. The methods described herein may be used in any combination with the other method described herein, including but not limited to, methods of identifying an ECM structure location, methods of removing a supernatant from a cell pellet, methods of fragmenting cell clusters, and methods of seeding a solution of cells.

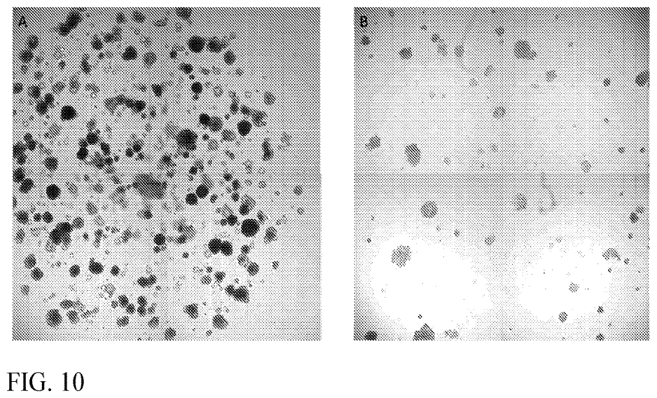

FIG. 10 is a micrograph showing comparison of a well before (left) and after (right) removal from ECM as described herein. Most of the cells have been recovered. What is left are the single layer cells that are not as dense as other cell clusters and sticky to the surface of the cell culture plate.

In methods described herein, the x,y coordinates may be at or near the x.y center position of the ECM structure. For example, the pipette tip may be positioned at the center of the ECM structure, or within 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, or within 0-2 mm, or 0.01-1.5 mm of the center of the x,y center of the ECM structure.

Methods may include moving the pipette tip through the dome in a multiplicity of steps, each step comprising an X, Y predetermined position on a cell culture plate to disrupt/break the ECM structure and optionally liquify the hydrogel dome to free the target cells from the ECM structure. The pipette tip may be aspirated and/or the contents of the pipette tip dispense at one or more, two or more, a multiplicity of, or each of the predetermined positions.

Each of the X, Y predetermined positions within the well may be calculated comprising:

X(stepnumber)=Xwellcenter+alpha/steps*stepnumber*COS(theta/steps*stepnumber); and

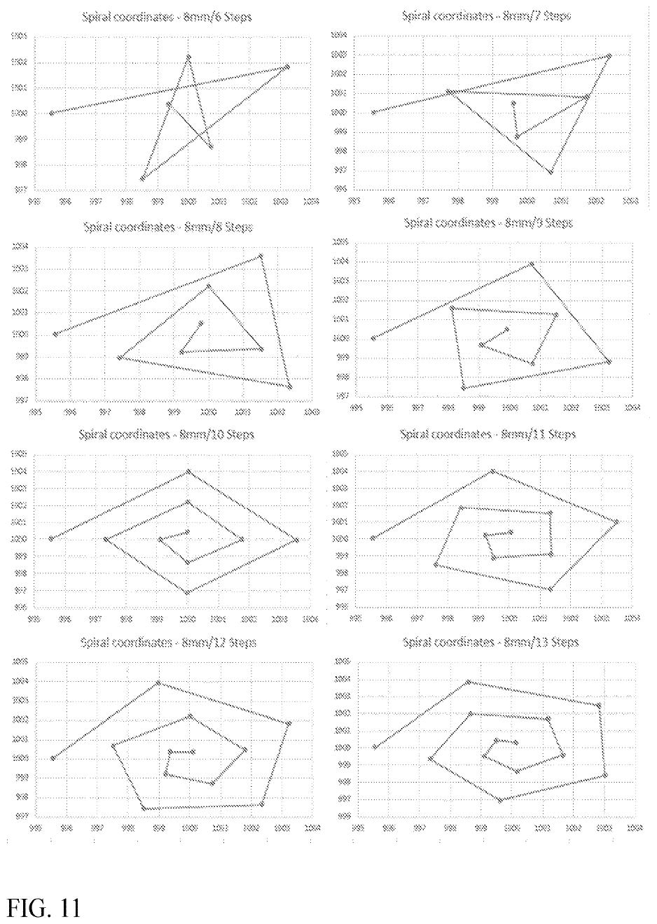

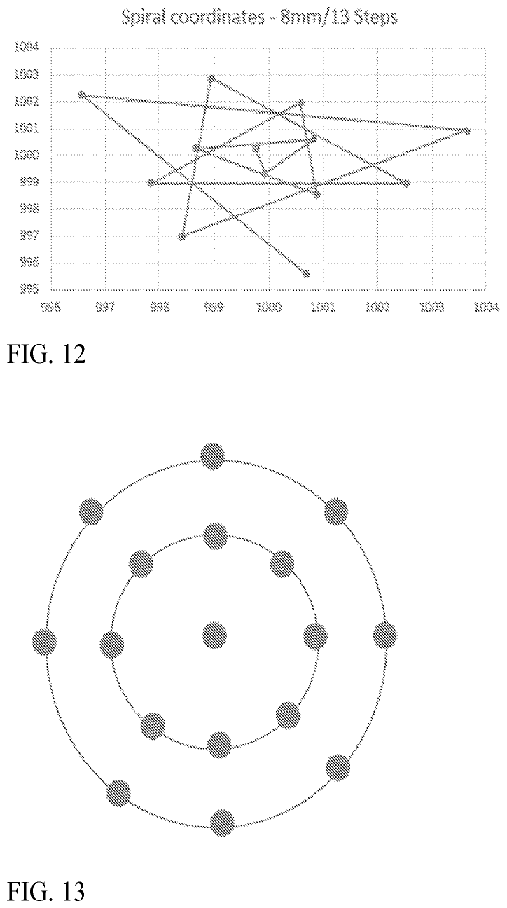

Y(stepnumber)=Ywellcenter+alpha/steps*stepnumber*SIN(theta/steps*stepnumber),

wherein alpha=ECM structure diameter (mm)/1.8; steps=number of steps for moving the pipette tip through the ECM structure; Xwellcenter=x position of ECM structure center on the surface of a culture plate; Ywellcenter=y position of ECM structure center on the surface of the culture plate; and theta=a constant value between 5 and 50. In some cases, the number of steps is an integer from 2-20, 3-18, 4-17, 5-15, 6-12, or 8-10. In some cases, theta is a constant from 5-50, 10-45, 15-40, 15-35, or any number in between. In some cases, theta, is selected from the group consisting of 5, 10, 12, 13, 14, 15, 15.7, 16, 17, 18, 20, 22, 24, 26, 28, 30, 32, 34, 36, 38, 40, 45, or 50, or any number in between. The x,y center position of the ECM structure in the well may be determined based on the original seeding position in the well, volume of ECM structure deposited in the well, and/or imaging of the ECM structure. The pipette tip may be used to puncture the ECM structure, and/or aspirate and/or dispense liquid within the ECM structure. For example, the pipette tip may be used to apply, remove, dispense, or aspirate a liquid or gas (for example air) within the ECM structure. Inputs to the system may include seeding position x,y coordinates in the well and the known volume of the ECM structure. Another input may be the number of steps of ECM structure disruptions or pipette aspirations/dispensings. The diameter of the ECM structure may be calculated based on the volume. The volume of an ECM structure may be determined in any way. For example, the volume may be based on the volume of ECM that was deposited to form the ECM structure, or by ascertaining on or more of the overall shape, the height, and circumference of the ECM structure. The diameter of the ECM structure may be determined by imaging. The diameter of the ECM structure may be any appropriate diameter. For example, the diameter of the ECM structure may be 1-12 mm, 2-10 mm, 3-8 mm, or 3-6 mm. The number of steps may be any appropriate number. For example, the number of steps may be 1, 2, 3, 4, 5, 6, 7, 8, 9, 10, 11, 12, 13, 14, 15, 16, 17, 18, 19, or 20, or 2-20, 3-18, 4-17, 5-15, 6-12, or 8-10. For example, inputs include the diameter of a hydrogel dome in this case 8 mm, the number of steps is 8, and well center position X, Y coordinates. In some cases, the number of steps/positions may be minimized to effectively break the hydrogel in order to minimize process time. The output may include an automated sequence/pattern of puncturing/aspirating positions within the dome with a pipette by the liquid handler. In some embodiments, the pattern of the puncturing/aspirating positions may be a spiral pattern. In some embodiments, the pattern of the puncturing/aspirating positions is a star pattern. In some embodiments, the pattern of the puncturing/aspirating positions is a zig-zag pattern. At each position, the automated pipette may aspirate within the dome. The pipette tip may be dragged beneath the surface of the dome between steps/positions. For example, the pipette tip position in the Z axis may be maintained below the surface of the hydrogel dome between steps/positions for efficient breaking of the dome. Between steps, the pipette tip position in the Z axis may be maintained at or near the bottom of the hydrogel dome. For example, the pipette tip may be maintained below the surface, within 0.5, 1.0, 1.5, or 2 mm of the surface, at or within 0.5, 1.0, 1.5, or 2 mm near the center of the Z axis, at the bottom, or within 0.5, 1.0, 1.5, or 2 mm of the bottom of the dome between steps or during aspirating. The Z axis position may be determined by the volume of the ECM structure or by imaging the cell culture surface. Exemplary FIG. 11 shows an output series of spiral X, Y coordinates of the automated pipettor within the well using inputs of well center position, 8 mm dome diameter, and 6-13 steps, using theta of 15.7 (FIG. 12) or 30 (FIG. 12) resulting in effective breaking of a hydrogel dome. For example, the solid hydrogel can be transformed into a liquid, thereby separating the cells from the hydrogel 106. The ECM structure may be transformed into a liquid by, e.g., harsh pipetting, or shear forces of a liquid handler, with or without a decrease in temperature, for example, to about 4 deg C. to about 10 deg C., or about 10 deg C., from incubation temperature (about 37 deg C.). A dissociation reagent can be added in order to dissociate cell clusters such as organoids into stem cells and a plurality of additional cell types.

In particular embodiments, the breaking points include a central breaking point in the center of the ECM structure and a number of other breaking points arranged radially around the central breaking point. Where required by the size of the ECM structure, multiple concentric radial arrangements of breaking points may be arranged around the central breaking point. In particular embodiments, there may be 1, 2, 3, or more concentric radial arrangements. An example of disruption points with two concentric radial arrangements is provided in FIG. 13.

In some embodiments, the radius of the radial arrangements of disruption points is adjustable. The flexibility to change the radius of the circles ensures that ECM structures with different sizes (volumes) may be efficiently disrupted and the multiple concentric radial arrangements design ensures that ECM structures with large size may be efficiently disrupted.

In embodiments, the x,y positions of multiple ECM structures may be known. Where multiple ECM structures are present on the surface of a culture plate, imaging as described herein or other techniques may be used to determine a pattern of x.y predetermined positions located in more than one or all of the ECM structures on the culture plate. Such patterns include radial arrangements, spiral patterns, star patterns, zig-zag patterns and combinations thereof Movement of the pipette tip to each of the x,y predetermined positions may then disrupt more than one or all of the ECM structures on the culture plate.

In an embodiment, the x,y locations of multiple ECM structures on the surface of a cell culture plate are determined as described herein. A substantially automated process may be used to create a pattern for the disruption of one or more or all of the ECM structures. Various combinations of patterns may be used to disrupt the ECM structures. In an example of such an embodiment, the pipette tips proceeds to each of multiple ECM structures in a spiral pattern, and while at each of the ECM structures, the pipette tip moves through a star pattern of x,y predetermined positions within each ECM structure so as to disrupt it before continuing the radial pattern to the next ECM structure.

Methods may further include disrupting the extracellular matrix structure by dispensing of a liquid, such as a culture medium, into at least one position within the extracellular matrix at a fixed height above the bottom of the culture plate and at a fixed speed.

In certain embodiments, the aspirating/dispensing of the liquid is performed by an automated/computer-controlled liquid handler/pipette/pipettor. In embodiments, the entire method of removal of cells from an extracellular matrix structure is automated and/or performed without human intervention. In particular embodiments, the method is carried out by/within an automated cell culture apparatus.

In some embodiments, the height the pipette is held above the surface of the culture plate is referred to as a fixed height. In some embodiments, the fixed height is between about 1 mm and about 3 mm above the bottom of the culture plate on which the ECM structure is supported. In some embodiments, the fixed height is about 2 mm above the bottom of the culture plate. In particular embodiments the fixed height is 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 mm above the bottom of the culture plate. In some embodiments, the liquid is dispensed at speed between about 400 μL/s and about 600 μL/s. In some embodiments, the culture medium is dispensed at speed of about 500 μL/s. In particular embodiments, the culture medium is dispensed at speed of 400, 410, 420, 430, 440, 450, 460, 470, 480, 490, 500, 510, 520, 530, 540, 550, 560, 570, 580, 590, or 600 μL/s. In some embodiments, a cell dissociation reagent is added to the center of the well. In some embodiments, the cell dissociation reagent is added at a fixed height between about 0.05 mm and about 0.3 mm above the bottom of the surface of the culture plate. In some embodiments, the cell dissociation reagent is added at a fixed height about 0.1 mm above the bottom of the surface of the culture plate

In a particular embodiment, the method includes the following steps: 1) Aspirate culture medium (500 μL) from the culture plate using 1000 μL tip along the side of the well at a fixed height 0 mm from bottom. A sequence of x,y coordinate aspiration positions are pre-setup along the side of the wells to avoid aspirating the ECM structures, which congregate in the center of the wells. 2) Add 500 μL Gentle Cell Dissociation Reagent or Corning Cell Recovery Solution to the center of the well at a fixed height 0.1 mm from bottom. 3) Wait for 60 s before proceeding to the next steps. 4) A sequence of x,y predetermined positions is configured for subsequent use (see e.g FIGS. 11-13) The 500 μL of the solution is aspirated from the side of the well at a fixed position 0 mm above bottom and dispensed to one or more of the x,y predetermined positions at a speed 500 ul/s (fastest speed) at a fixed height 2 mm above the bottom to break the ECM structures. This step loops until all x,y predetermined positions have been covered. 6) The plate is tilted at 10°. 7) The 500 μL of the cell solution is aspirated at a fixed height 0.1 mm above bottom and transfer to the well in a 96 deep well plate at a fixed height 0.5 mm above bottom. 8) The plate is returned to the horizontal position. 9) Steps 2, 5, 6, and 7 are repeated for a second wash and any residual cells are collected.

Removal of supernatant from a cell pellet. Described herein are methods for removal of supernatant from a cell pellet. The method includes centrifuging a cell solution in a container to form a cell pellet and a supernatant; aspirating a first volume of the supernatant at a first fixed height from the bottom of the container and a first fixed speed; and aspirating a second volume of the supernatant at a second fixed height from the bottom of the container and a second fixed speed. The methods described herein may be used in any combination with the other method described herein, including but not limited to, methods of identifying an ECM structure location, methods of removing cells from ECM, methods of fragmenting cell clusters, and methods of seeding a solution of cells.

In embodiments, the cell pellet comprises organoids. In particular embodiments, the organoids are obtained by the removal of cells (e.g. organoids) from an ECM structure as described herein. In certain embodiments, the aspirating and dispensing of the culture medium is performed by an automated/computer-controlled liquid handler/pipette/pipettor. In embodiments, the entire method of removal of supernatant from a cell pellet is automated and/or performed without human intervention. In particular embodiments, the method is carried out by/within an automated cell culture apparatus.

In some embodiments, the pipette is held at a first height above the bottom of the container for aspiration of a first volume of the supernatant, and moved to a lower height for aspiration of a second volume of the supernatant. This variable pipetting speed scheme is designed to ensure that as much supernatant as possible is aspirated while minimizing loss of pellet in the bottom of the container. In certain embodiments, the first volume of supernatant may be discarded or retained in the pipette prior aspirating the second volume of supernatant.

In some embodiments, the first fixed height is between about 20 mm and 40 mm. In some embodiments, the first fixed height is about 30 mm. In particular embodiments, the first fixed height is 20, 21, 22, 23, 24, 25, 26, 27, 28, 29, 30, 31, 32, 33, 34, 35, 36, 37, 38, 39, or 40 mm. In some embodiments, the second fixed height is between about 2 mm and 8 mm. In some embodiments, the second fixed height is about 5 mm. In particular embodiments, the second fixed height is 2, 2.5, 3, 3.5, 4, 4.5, 5, 5.5, 6, 6.5, 7, 7.5, or 8 mm. In some embodiments, the first volume is between about 400 μL and about 600 μL. In particular embodiments the first volume is 400, 410, 420, 430, 440, 450, 460, 470, 480, 490, 500, 510, 520, 530, 540, 550, 560, 570, 580, 590, or 600 μL. In some embodiments, the first volume is about 500 μL. In some embodiments, the second volume is between about 300 μL and about 500 μL. In some embodiments, the second volume is about 400 μL. In particular embodiments, the second volume is 300, 310, 320, 330, 340, 350, 360, 370, 380, 390, 400, 410, 420, 430, 440, 450, 460, 470, 480, 490, or 500 μL.

The computer-controlled pipettes are capable of aspirating and dispensing liquids at various speeds. Aspirating at too high a speed, too close to the organoids may result in the loss of those organoids so the speed of aspiration has been carefully determined for minimum damaging results. In some embodiments, the first speed is between about 200 μL/s and about 300 μL/s. In some embodiments, the first speed is about 250 μL/s. In particular embodiments, the first speed is 200, 205, 210, 215, 220, 225, 230, 235, 240, 245, 250, 255, 260, 265, 270, 275, 280, 285, 290, 295, or 300, μL/s. In some embodiments, the second speed is between about 35 μL/s and about 65 μL/s. In some embodiments, the second speed is about 50 μL/s. In particular embodiments, the second speed is 35, 40, 45, 50, 55, 60, or 65 μL/s

In certain embodiments, the container is a well in a 6, 12, 24, or 96 well plate.

Fragmentation of cell clusters. Described herein are methods for the fragmentation of cell clusters. Nonlimiting examples of cell clusters include organoids, spheroids, and tumeroids. The methods include, in a container comprising a cell cluster pellet and a supernatant of a first volume, a) aspirating and dispensing a second volume at a first fixed height from the bottom of the container multiple times sequentially; b) aspirating a third volume at a second fixed height from the bottom of the container and dispensing the third volume back into the container; and c) repeating the steps a) and b). The methods described herein may be used in any combination with the other method described herein, including but not limited to, methods of identifying an ECM structure location, methods of removing cells from ECM, methods of removing a supernatant from a cell pellet, and methods of seeding a solution of cells.

In embodiments, the cell clusters are obtained by the removal of cell clusters (e.g. organoids) from an ECM structure as described herein. In certain embodiments, the aspirating and dispensing of the culture medium is performed by an automated/computer-controlled liquid handler/pipette/pipettor. In embodiments, the entire method of fragmentation of cell clusters is automated and/or performed without human intervention. In particular embodiments, the method is carried out by/within an automated cell culture apparatus.

In some embodiments, the first volume is between about 75 μL and about 125 μL. In some embodiments, the first volume is about 100 μL. In particular embodiments, the first volume is 75, 80, 85, 90, 95, 100, 105, 110, 115, 120, or 125 μL.

In some embodiments, the first fixed height is between about 0.1 mm and about 0.3 mm. In some embodiments, the first fixed height is about 0.2 mm. In particular embodiments, the first fixed height is 0.1, 0.11, 0.12, 0.13, 0.14, 0.15, 0.16, 0.17, 0.18, 0.19. 0.2, 0.21, 0.22, 0.23, 0.24, 0.25, 0.26, 0.27, 0.28, 0.29, or 0.3 mm. In some embodiments, the second volume is between about 70 μL and about 90 μL. In some embodiments, the second volume is about 80 μL. In particular embodiments, the second volume is 70, 71, 72 73, 74, 75, 76, 77, 78, 79, 80, 81, 82, 83, 84 85, 86, 87, 88, 89, or 90 μL.

In some embodiments, the second fixed height is between about 0.1 mm and about 0.3 mm. In some embodiments, the second fixed height is about 0.2 mm. In particular embodiments, the second fixed height is 0.1, 0.11, 0.12, 0.13, 0.14, 0.15, 0.16, 0.17, 0.18, 0.19. 0.2, 0.21, 0.22, 0.23, 0.24, 0.25, 0.26, 0.27, 0.28, 0.29, or 0.3 mm. In some embodiments, the third volume is between about 75 μL and about 105 μL. In some embodiments, the third volume is about 90 μL. In particular embodiments, the third volume is 75, 76, 77, 78, 79, 80, 81, 82, 83, 84 85, 86, 87, 88, 89, 90, 91, 92, 93, 94, 95, 96, 97, 98, 99, 100, 101, 102, 103, 104, or 105 μL.

In some embodiments, in step a), the second volume is aspirated and dispensed between about 40 times and about 60 times. In some embodiments, in step a), the second volume is aspirated and dispensed about 50 times. In particular embodiments, in step a), the second volume is aspirated and dispensed 40, 41, 42, 43, 44, 45, 46, 47, 48, 49, 50, 51, 52, 53, 54, 55, 56, 57 58, 59, or 60 times.

In some embodiments, steps a) and b) are repeated between two and ten times. In some embodiments, steps a) and b) are repeated four times. In particular embodiments, steps a) and b) are repeated 1, 2, 3, 4, 5, 6, 7, 8, 9, or 10 times.

In certain embodiments, the container is a well in a 6, 12, 24, or 96 well plate.

In a particular embodiment, fragmenting the organoids comprises 1) providing a 100 μL organoids solution. 2) The cell cluster pellet is fragmented by pipetting 80 μL at a fixed height 0.2 mm from the bottom 50 times before 90 μL is aspirated at a fixed height 0.2 mm from the bottom and dispensed back to the well, using 300 μL pipette tips. 3) Step 15 is repeated 4 times, which results in around total 200 pipette times.

Fragmentation of the bulk cell clusters aids in even distribution of the cell clusters in the passage containers. In some embodiments, the pipette tips are 300 μL tips. The 300 μL tips are used to assist in the fragmentation of the bulk cell clusters. In other embodiments, other sizes of pipette tips are used, for example in some embodiments, 1000 μL pipette tip are used. Cell cluster fragmentation may be carried out after the two rounds of centrifugation and supernatant removal to improve the organoid recovery. This step requires a balance between over and under fragmentation of the cell clusters. Compared to manual passaging that require repeated fragment size check using the microscope, the overall fragment size distribution of cell clusters may be standardized with regards to the total pipetting times.

Seeding of a solution of cells. Described herein are methods for seeding of a solution of cells. The method includes a) mixing, via aspirating and dispensing, about 60% of the volume of the solution of cells multiple times; b) aspirating a portion of the mixed solution to provide an aspirated solution and remaining non-aspirated solution; c) dispensing at least a portion of the aspirated solution to a new culture plate; and d) repeating steps a) through c) with the remaining non-aspirated solution. The methods described herein for the seeding of cells may be performed in a substantially automated process. For example, one or more of steps a) through c) above may be performed by an automated liquid handler with minimal or no human intervention. The methods described herein may be used in any combination with the other methods described herein, including but not limited to, methods of identifying an ECM structure location, methods of removing cells from ECM, methods of fragmenting cell clusters, and methods of removing a supernatant from a cell pellet.

FIG. 15 is micrographs showing the growth of the organoids that are passaged and seeded via liquid handler at (A) Day 1, (B) Day 2, (C) Day 5 and (D) Day 7.

In embodiments, the solution of cells may comprise cell clusters or fragments thereof. In embodiments, the organoids are obtained by the removal of cells (e.g. organoids) from an ECM structure as described herein. In further embodiments, the fragments of cell clusters may be obtained by methods of cell cluster fragmentation described herein. In certain embodiments, the aspirating and dispensing of the culture medium is performed by an automated/computer-controlled liquid handler/pipette/pipettor. In embodiments, the entire method of seeding of a solution of cells is automated and/or performed without human intervention. In particular embodiments, the method is carried out by/within an automated cell culture apparatus.

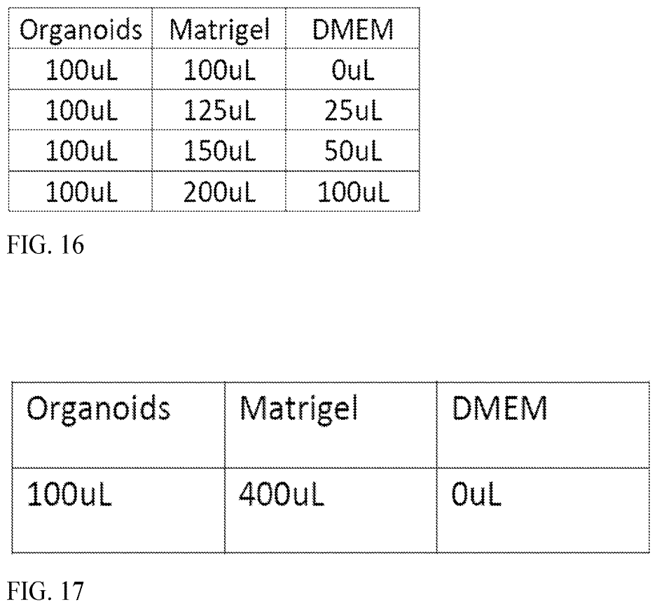

In embodiments, the solution of cells comprises an extracellular matrix. In particular embodiments, the extracellular matrix is Matrigel or other hydrogel that mimics an organ's native microenvironment. The amount of Matrigel or other hydrogel may be varied to provide different ECM concentrations for seeding. For example, FIG. 16 provides the components for creating 50% Matrigel ECM structures. In a further example, FIG. 17 provides the components for creating 80% Matrigel ECM structures.

In some embodiments, the mixing via aspirating and dispensing is conducted at a fixed height of between about 2 mm and about 4 mm from the bottom of a container in which the solution of cells is located. In embodiments, the mixing via aspirating and dispensing is conducted at a fixed height of about 3 mm from the bottom of a container in which the solution of cells is located. In particular embodiments, the mixing via aspirating and dispensing is conducted at a fixed height of 2, 2.1, 2.2, 2.3, 2.4, 2.5, 2.6, 2.7, 2.8, 2.9, 3, 2.1, 3.2, 3.3, 3.4, 3.5, 3.6, 3.7, 3.8, 3.9, or 4 mm from the bottom of a container in which the solution of cells is located. In certain embodiments, the mixing via aspirating and dispensing is conducted at least 5 times.

In embodiments, when conducting step d), the aspirating and dispensing is conducted at a fixed height from the bottom of a container in which the solution of cells is located that is smaller than the previous round of steps a) and b). In particular embodiments, step d) is repeated until the seeding of the solution of cells is complete and each time step d) is performed, the aspirating and dispensing of step a) is conducted a fixed height that is small than the immediately proceeding round of steps a) and b). In a more particular embodiment, each time step d) is performed, the aspirating and dispensing of step a) is conducted a fixed height that is ⅓ smaller than the immediately preceding round of steps a) and b).

In embodiments, in step c), dispensing at least a portion of the aspirated solution to a culture plate not previously used to culture the cells comprises dispensing the aspirated solution at a distance of between about 0.1 mm and about 0.5 mm from the surface of the culture plate. In embodiments, in step c), dispensing at least a portion of the aspirated solution to a culture plate not previously used to culture the cells comprises dispensing the aspirated solution at a distance of about 0.3 mm from the surface of the culture plate. In particular embodiments, in step c), dispensing at least a portion of the aspirated solution to a culture plate not previously used to culture the cells comprises dispensing the aspirated solution at a distance o, 0.1, 0.11, 0.12, 0.13, 0.14, 0.15, 0.16, 0.17, 0.18, 0.19. 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49. or 0.5 mm

In some embodiments, in step c), dispensing at least a portion of the aspirated solution to a culture plate not previously used to culture the cells comprises dispensing the aspirated solution at between about 5 μL/s and about 9 μL/s. In some embodiments, in step c), dispensing at least a portion of the aspirated solution to a culture plate not previously used to culture the cells comprises dispensing the aspirated solution at about 7 μL/s. In particular embodiments, in step c), dispensing at least a portion of the aspirated solution to a culture plate not previously used to culture the cells comprises dispensing the aspirated solution at 5, 5.1, 5.4, 5.6, 5.8, 6, 6.2, 6.4, 6.6, 6.8, 7, 7.2, 7.4, 7.6, 7.8, 8, 8.2, 8.4, 8.6, 8.8, or 9 μL/s.

To achieve uniform seeding of the cell clusters or fragments thereof, it is often beneficial to mix the organoids in the solution. A small pipette tip often aids in mixing the cell clusters or fragments thereof in the solution. In particular embodiments, a 300 μL tip is used to do seeding with the benefit of large volume capacity to do mixing and small tip opening to do small volume seeding.

In some embodiments, an ECM/cell cluster fragment solution is mixed utilizing aspiration and dispensing of 0.6*Vremaining, with Vremaining up to 500 μL being the remaining volume, to ensure uniform seeding. In certain embodiments, an extra 40 μL is aspirated for seeding in addition to the required volume with the formula Vtotal=nseeding*Vseeding+40 μL, with nseeding being the number of seeding per round and Vseeding being the volume of dome per seeding to ensure enough solution for the last seeding in one round. In certain embodiments, 10 μL is pre-dispensed back into the solution of cells to ensure successful small-volume seeding (like 7 ul) in the first well during one-round seeding. In certain embodiments, the aspiration of step b) occurs at 3 mm from the bottom when Vremaining>=2*Vtotal and at 1 mm from the bottom when Vremaining<2*Vtotal to enable uniform cell cluster seeding in the destination plate and reduce the dead volume.

In a particular embodiment, the method is carried out as follows: a 96 deep well plate with 500 μL total Matrigel/cell cluster solution per well and 2 seedings per round to one or more 24-well destination plate as an example. Mix at a fixed height of 3 mm from the bottom with 300 μL for 5 cycles before aspirating 120 μL at a fixed height of 3 mm from the bottom, using 300 μL tips. Dispense 10 μL back to the 96 deep plate at a low speed of 7 μL/s to avoid any bubbles. Dispense 40 μL to the well of the destination plate at a fixed height 0.3 mm from the bottom with a low speed of 7 μL/s. Loops until one round of 2 wells are completed. Discard the tips with the remaining Matrigel/cell cluster solution. When looping over the rest of the wells in the destination plate, we mix with 60% of the volume of the remaining Matrigel/cell cluster solution in the 96 deep plate before aspiration. When the volume of the remaining Matrigel/cell cluster solution is less than 240 μL, we aspirate at a lower height of 1 mm from the bottom.

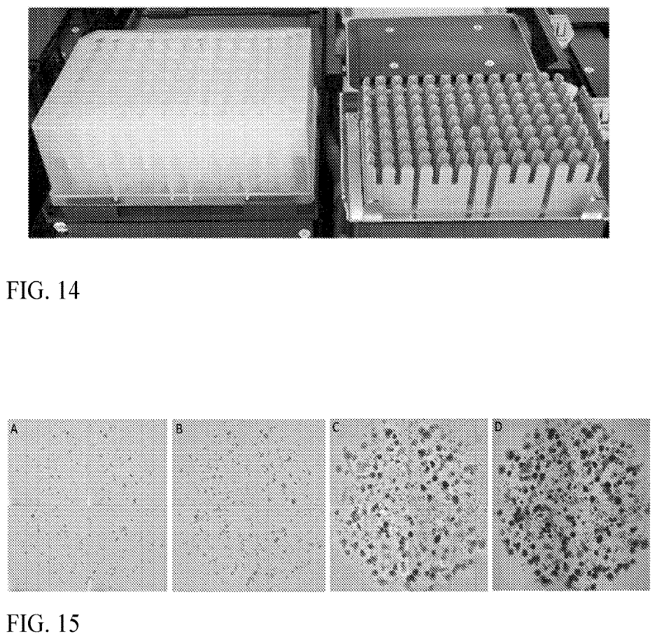

In all embodiments, 96 well deep plates may be used for the liquid handling. Instead of using 15 ml tube or 2 ml tube that require decapping, a 96 deep well plate for liquid handler may be used. There are two purposes here: 1) 96 deep well plate is more automation friendly for delidding and transport, while tubes require decapping, that needs special tool. 2) Additionally, an adapter for the 96 deep well plate enables cooling of the plate at 4° C., as required for Matrigel (FIG. 14).

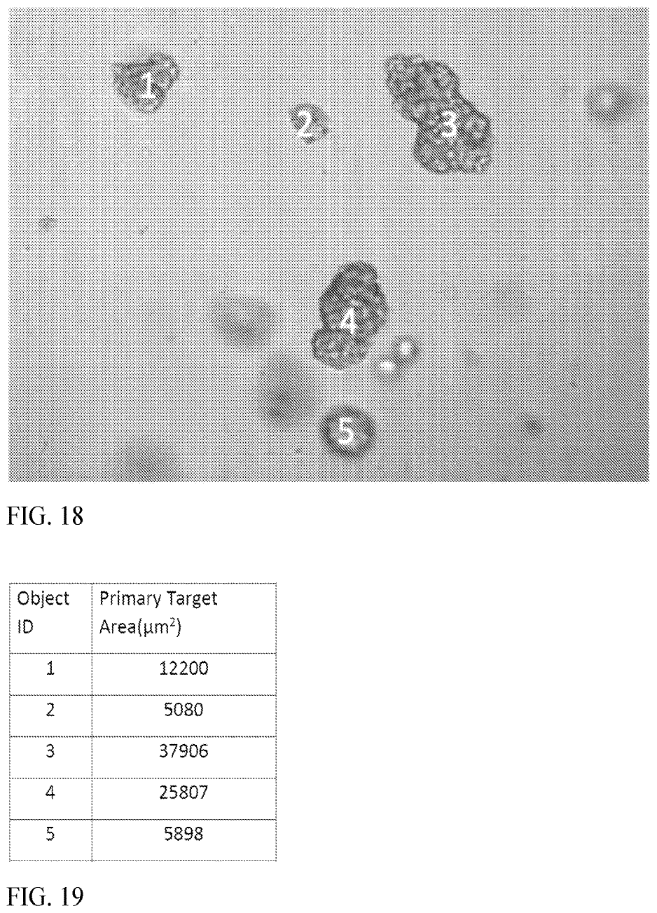

Organoid Recovery Rate Analysis: IN Carta software is used as a tool to develop the analysis workflow that quantifies the area of the organoids, which is then used to calculate the organoid recovery rate after passaging. Examples are shown in FIG. 18 with corresponding values of area in FIG. 19.

The methods described herein may be used in any combination with the other method described herein. Such methods include:
A) methods of identifying an ECM structure location,
B) methods of removing cells from ECM,
C) methods of removing a supernatant from a cell pellet,
D) methods of fragmenting cell clusters, and
E) methods of seeding a solution of cells.

Described herein are methods including, as defined above, A and B. Described herein are methods including, as defined above, A and C. Described herein are methods including, as defined above, A and D. Described herein are methods including, as defined above, A and E. Described herein are methods including, as defined above, B and C. Described herein are methods including, as defined above, B and D. Described herein are methods including, as defined above, B and E. Described herein are methods including, as defined above, C and D. Described herein are methods including, as defined above, C and E. Described herein are methods including, as defined above, D and E. Described herein are methods including, as defined above, A, B, and C. Described herein are methods including, as defined above, A, B, and D. Described herein are methods including, as defined above, A, B, and E. Described herein are methods including, as defined above, A, C, and D. Described herein are methods including, as defined above, A, C, and E. Described herein are methods including, as defined above, A, D, and E. Described herein are methods including, as defined above, B, C, and D. Described herein are methods including, as defined above, B, C, and E. Described herein are methods including, as defined above, B, D, and E. Described herein are methods including, as defined above, C, D, and E. Described herein are methods including, as defined above, A, B, C, and D. Described herein are methods including, as defined above, A, B, C, and E. Described herein are methods including, as defined above, A, C, D, and E. Described herein are methods including, as defined above, B, C, D, and E.

As will be appreciated by one of ordinary skill in the art, various aspects of the present technology may be embodied as a system, method, or computer program product. Accordingly, some aspects of the present technology may take the form of an entirely hardware embodiment, an entirely software embodiment (including firmware, resident software, micro-code, etc.), or a combination of hardware and software aspects that may all generally be referred to herein as a circuit, module, system, and/or network. Furthermore, various aspects of the present technology may take the form of a computer program product embodied in one or more computer-readable mediums including computer-readable program code embodied thereon.

Any combination of one or more computer-readable mediums may be utilized. A computer-readable medium may be a computer-readable signal medium or a physical computer-readable storage medium. A physical computer readable storage medium may be, for example, but not limited to, an electronic, magnetic, optical, crystal, polymer, electromagnetic, infrared, or semiconductor system, apparatus, or device, etc., or any suitable combination of the foregoing. Non-limiting examples of a physical computer-readable storage medium may include, but are not limited to, an electrical connection including one or more wires, a portable computer diskette, a hard disk, random access memory (RAM), read-only memory (ROM), an erasable programmable read-only memory (EPROM), an electrically erasable programmable read-only memory (EEPROM), a Flash memory, an optical fiber, a compact disk read-only memory (CD-ROM), an optical processor, a magnetic processor, etc., or any suitable combination of the foregoing. In the context of this document, a computer-readable storage medium may be any tangible medium that can contain or store a program or data for use by or in connection with an instruction execution system, apparatus, and/or device.

Computer code embodied on a computer-readable medium may be transmitted using any appropriate medium, including but not limited to, wireless, wired, optical fiber cable, radio frequency (RF), etc., or any suitable combination of the foregoing. Computer code for carrying out operations for aspects of the present technology may be written in any static language, such as the C programming language or other similar programming language. The computer code may execute entirely on a user's computing device, partly on a user's computing device, as a stand-alone software package, partly on a user's computing device and partly on a remote computing device, or entirely on the remote computing device or a server. In the latter scenario, a remote computing device may be connected to a user's computing device through any type of network, or communication system, including, but not limited to, a local area network (LAN) or a wide area network (WAN), Converged Network, or the connection may be made to an external computer (e.g., through the Internet using an Internet Service Provider).

Various aspects of the present technology may be described above with reference to flowchart illustrations and/or block diagrams of methods, apparatus, systems, and computer program products. It will be understood that each block of a flowchart illustration and/or a block diagram, and combinations of blocks in a flowchart illustration and/or block diagram, can be implemented by computer program instructions. These computer program instructions may be provided to a processing device (processor) of a general purpose computer, special purpose computer, or other programmable data processing apparatus to produce a machine, such that the instructions, which can execute via the processing device or other programmable data processing apparatus, create means for implementing the operations/acts specified in a flowchart and/or block(s) of a block diagram.

Some computer program instructions may also be stored in a computer-readable medium that can direct a computer, other programmable data processing apparatus, or other device(s) to operate in a particular manner, such that the instructions stored in a computer-readable medium to produce an article of manufacture including instructions that implement the operation/act specified in a flowchart and/or block(s) of a block diagram. Some computer program instructions may also be loaded onto a computing device, other programmable data processing apparatus, or other device(s) to cause a series of operational steps to be performed on the computing device, other programmable apparatus or other device(s) to produce a computer-implemented process such that the instructions executed by the computer or other programmable apparatus provide one or more processes for implementing the operation(s)/act(s) specified in a flowchart and/or block(s) of a block diagram.

A flowchart and/or block diagram in the above figures may illustrate an architecture, functionality, and/or operation of possible implementations of apparatus, systems, methods, and/or computer program products according to various aspects of the present technology. In this regard, a block in a flowchart or block diagram may represent a module, segment, or portion of code, which may comprise one or more executable instructions for implementing one or more specified logical functions. It should also be noted that, in some alternative aspects, some functions noted in a block may occur out of an order noted in the figures. For example, two blocks shown in succession may, in fact, be executed substantially concurrently, or blocks may at times be executed in a reverse order, depending upon the operations involved. It will also be noted that a block of a block diagram and/or flowchart illustration or a combination of blocks in a block diagram and/or flowchart illustration, can be implemented by special purpose hardware-based systems that may perform one or more specified operations or acts, or combinations of special purpose hardware and computer instructions.

A first aspect includes an automated method for identifying the location of an extracellular matrix structure, the method comprising: using a substantially automated process to identify the X, Y position of the extracellular matrix structure on the surface of a culture plate.

A second aspect includes using a substantially automated process to disrupt an extracellular matrix structure at a predetermined X, Y position of the extracellular matrix structure on the surface of the culture plate with a pipette tip associated with a liquid handler.

A third aspect includes an automated method for identifying the location of and disrupting an extracellular matrix structure, the method comprising: using a substantially automated process to identify an X,Y predetermined position within the extracellular matrix structure on a surface of a culture plate; and using a substantially automated process to disrupt the extracellular matrix structure at the X,Y predetermined position of the extracellular matrix structure on the surface of the culture plate with a pipette tip associated with a liquid handler.

A fourth aspect includes prior to disrupting, identifying the location of the of the extracellular matrix structure on the surface of the culture plate with a substantially automated process.

A fifth aspect includes the extracellular matrix structure of any aspect enumerated herein being in the form of a dome.

A sixth aspect includes the extracellular matrix structure of any aspect enumerated herein being a hydrogel.

A seventh aspect includes the X,Y predetermined position of any aspect enumerated herein being at or near the center of the extracellular matrix structure.

An eighth aspect includes the X,Y predetermined position of the extracellular matrix structure of any aspect enumerated herein being determined by one or more parameters selected from the group consisting of the original seeding position on the surface of the culture plate and imaging of the extracellular matrix structure on the surface of the culture plate.

A ninth aspect includes a diameter of the extracellular matrix structure of any aspect enumerated herein being determined by a volume of the extracellular matrix deposited on the surface of the culture plate or imaging of the extracellular matrix structure on the surface of a culture plate.

A tenth aspect includes, in any aspect enumerated herein, moving the pipette tip in a multiplicity of steps to free the cells from the extracellular matrix structure, each of the multiplicity of moving steps comprising movement to an additional X,Y predetermined position within the extracellular matrix structure.

An eleventh aspect includes the surface of a culture plate of any aspect enumerated herein being a cell culture well.

A twelfth aspect includes wherein the culture plate of any aspect enumerated herein being a primary well of a well unit of a separation well microplate.

A thirteenth aspect includes, in any aspect enumerated herein, calculating each of the X,Y predetermined positions and any additional X,Y predetermined positions on the cell culture surface via:

$$X ( stepnumber ) = Xwellcenter + alpha / steps * stepnumber * COS ( theta / steps * stepnumber ) ;$$

$$and$$

$$Y ( stepnumber ) = Ywellcenter + alpha / steps * stepnumber * SIN ( theta / steps * stepnumber ) ,$$

$$wherein$$

$$alpha = extracellular matrix structure diameter ( mm ) / 1.8 ;$$

$$steps = number of steps for moving the pipette tip through the extracellular matrix structure , optionally an integer from 2 to 20 ;$$

$$Xwellcenter = x position of dome center within the well ;$$

$$Ywellcenter = y position of dome center within the well ;$$

$$and$$

$$theta = a constant value between 5 and 50.$$

A fourteenth aspect includes, in any aspect enumerated herein, the number of predetermined positions being selected from 2-20, 3-18, 4-17, 5-15, 6-12, or 8-10 X,Y predetermined positions.

A fifteenth aspect includes, in any aspect enumerated herein, the X,Y predetermined positions and the additional X.Y positions forming a pattern selected from the group consisting of a spiral pattern, a star pattern, and a zig-zag pattern within and/or through the extracellular matrix structure.

A sixteenth aspect includes, in any aspect enumerated herein, the X,Y predetermined positions comprising at least 9 X,Y predetermined positions with 8 X,Y predetermined positions arranged in a circle a fixed radius from the center of the extracellular matrix.

A seventeenth aspect includes, in any aspect enumerated herein, the X,Y predetermined positions comprising at least 17 predetermined positions with two groups of 8 X, Y predetermined positions arranged in two circles of different radii.

An eighteenth aspect includes, in any aspect enumerated herein, each of the multiplicity of steps of moving the pipette tip individually comprising a selection from
moving the pipette tip through the extracellular matrix structure so as to break the extracellular matrix structure, and/or
removing the pipette tip from the extracellular matrix structure prior to moving.

A nineteenth aspect includes, in any aspect enumerated herein, the liquid handler
aspirating the pipette tip, and/or
dispensing a liquid medium from the pipette tip
within the extracellular matrix structure at one or more, two or more, a multiplicity of, or each of the X, Y predetermined positions.

A twentieth aspect includes, in any aspect enumerated herein, repeatedly aspirating and dispensing one or more, two or more, a multiplicity of times at each X,Y predetermined position.

A twenty-first aspect includes, in any aspect enumerated herein, aspirating or dispensing at a fixed height above the bottom of the culture plate at a fixed speed.

A twenty-second aspect includes, in any aspect enumerated herein, the fixed height being between about 1 mm and about 3 mm above the culture plate surface.

A twenty-third aspect includes, in any aspect enumerated herein, the fixed height being about 2 mm above the culture plate surface.

A twenty-fourth aspect includes, in any aspect enumerated herein, the liquid medium being dispensed at speed between about 1 μL/s and about 600 μL/s.

A twenty-fifth aspect includes, in any aspect enumerated herein, the culture medium being dispensed at speed about 500 μL/s.

A twenty-sixth aspect includes, in any aspect enumerated herein, adding a cell dissociation reagent to the extracellular matrix structure.

A twenty-seventh aspect includes, in any aspect enumerated herein, adding a cell dissociation reagent to the extracellular matrix structure at a fixed height between about 0.05 mm and about 0.3 mm above the culture plate surface.

A twenty-ninth aspect includes, in any aspect enumerated herein, adding a cell dissociation reagent to the extracellular matrix structure at a fixed height about 0.1 mm above the culture plate surface.

A thirtieth aspect includes, in any aspect enumerated herein, aspirating the culture medium from the culture plate prior to dispensing the culture medium.

A thirty-first aspect includes, alone or in combination with any aspect enumerated herein,
centrifuging a cell solution in a container to form a cell pellet and a supernatant;
aspirating a first volume of the supernatant at a first fixed height from the bottom of the container and a first fixed speed; and
aspirating a second volume of the supernatant at a second fixed height from the bottom of the container and a second fixed speed.

A thirty-second aspect includes, in any aspect enumerated herein, the cell pellet comprising organoids.

A thirty-third aspect includes, in any aspect enumerated herein, the first volume being between about 400 μL and about 600 μL.

A thirty-fourth aspect includes, in any aspect enumerated herein, the first volume being about 500 μL.

A thirty-fifth aspect includes, in any aspect enumerated herein, the first fixed height being between about 20 mm and about 40 mm from the bottom.

A thirty-sixth aspect includes, in any aspect enumerated herein, the first fixed height being about 30 mm.

A thirty-seventh aspect includes, in any aspect enumerated herein, the first speed being between about 200 μL/s and about 300 μL/s.

A thirty-eighth aspect includes, in any aspect enumerated herein, the first speed being about 250 μL/s.

A thirty-ninth aspect includes, in any aspect enumerated herein, the second volume being between about 300 μL and about 500 μL.

A fortieth aspect includes, in any aspect enumerated herein, the second volume being about 400 μL.

A forty-first aspect includes, in any aspect enumerated herein, the second fixed height being between about 2 mm and about 8 mm.

A forty-second aspect includes, in any aspect enumerated herein, the second fixed height being about 5 mm.

A forty-third aspect includes, in any aspect enumerated herein, the second speed being between about 35 μL/s and about 65 μL/s.

A forty-fourth aspect includes, in any aspect enumerated herein, the second speed being about 50 μL/s.

A forty-fifth aspect includes, in any aspect enumerated herein, the container being a well in a 96 well plate.

A forty-sixth aspect includes, alone or in combination with any aspect enumerated herein, providing a container comprising an organoid pellet and a supernatant of a first volume,
a) aspirating and dispensing a second volume at a first fixed height from the bottom of the container multiple times sequentially;
b) aspirating a third volume at a second fixed height from the bottom of the container and dispensing the third volume back into the container; and
c) repeating the steps a) and b).

A forty-seventh aspect includes, in any aspect enumerated herein, the organoid pellet being obtained by the thirty-first aspect.

A forty-eighth aspect includes, in any aspect enumerated herein, the first volume being between about 75 μL and about 125 μL.

A forty-ninth aspect includes, in any aspect enumerated herein, the first volume being about 100 μL.

A fiftieth aspect includes, in any aspect enumerated herein, the second volume being between about 70 μL and about 90 μL.

A fifty-first aspect includes, in any aspect enumerated herein, the second volume being about 80 μL.

A fifty-second aspect includes, in any aspect enumerated herein, the first height being between about 0.1 mm and about 0.3 mm.

A fifty-third aspect includes, in any aspect enumerated herein, the first height being about 0.2 mm.

A fifty-fourth aspect includes, in any aspect enumerated herein, the third volume being between about 75 μL and about 125 μL.

A fifty-fifth aspect includes, in any aspect enumerated herein, the third volume being about 90 μL.

A fifty-sixth aspect includes, in any aspect enumerated herein, the second height being between about 0.1 mm and about 0.3 mm.

A fifty-seventh aspect includes, in any aspect enumerated herein, the second height being about 0.2 mm.

A fifty-eighth aspect includes, in any aspect enumerated herein, step a) comprising aspirating and dispensing the second volume between about 40 times and about 60 times.

A fifty-ninth aspect includes, in any aspect enumerated herein, the second volume being aspirated and dispensed about 50 times.

A sixtieth aspect includes, in any aspect enumerated herein, step c) being repeated between two and ten times.

A sixty-first aspect includes, in any aspect enumerated herein, step c) being repeated four times.

A sixty-second aspect includes, in any aspect enumerated herein, the container being a well in a 96 well plate.

A sixty-third aspect includes, alone or in combination with any aspect enumerated herein:
a) mixing, via aspirating and dispensing, about 60% of the volume of a solution of cells multiple times;
b) aspirating a portion of the mixed solution to provide an aspirated solution and remaining non-aspirated solution;
c) dispensing at least a portion of the aspirated solution to a culture plate not previously used to culture the cells; and
d) repeating steps a) through c) with the remaining non-aspirated solution.

A sixty-fourth aspect includes, in any aspect enumerated herein, the solution of cells comprising organoids or fragments thereof.

A sixty-fifth aspect includes, in any aspect enumerated herein, the solution of cells comprising an extracellular matrix.

A sixty-sixth aspect includes, in any aspect enumerated herein, the mixing via aspirating and dispensing being conducted at a fixed height of about 3 mm from the bottom of a container in which the solution of cells is located.

A sixty-seventh aspect includes, in any aspect enumerated herein, the mixing via aspirating and dispensing being conducted at a fixed height of between about 2 mm and about 4 mm from the bottom of a container in which the solution of cells is located.

A sixty-eighth aspect includes, in any aspect enumerated herein, the mixing via aspirating and dispensing being conducted at least five times.

A sixty-ninth aspect includes, in any aspect enumerated herein, conducting aspirating and dispensing of the remaining non-aspirated solution from the bottom of a container in which the solution of cells at a fixed height that is smaller than a previous round of aspirating and dispensing.

A seventieth aspect includes, in any aspect enumerated herein, conducting aspirating and dispensing of the remaining non-aspirated solution from the bottom of a container in which the solution of cells at a fixed height that is ⅓ the height of a previous round of aspirating and dispensing.

A seventy-first aspect includes, in any aspect enumerated herein, dispensing at least a portion of the aspirated solution to the second culture plate comprises dispensing the aspirated solution at a distance of about 0.3 mm from the surface of the culture plate.

A seventy-second aspect includes, in any aspect enumerated herein, dispensing the aspirated solution at a distance of between about 0.1 mm and 0.5 mm from the surface of the culture plate.

A seventy-third aspect includes, in any aspect enumerated herein, dispensing the aspirated solution at a rate of about 7 μL/s.

The invention has been described with reference to various specific and preferred embodiments and techniques. Nevertheless, it is understood that many variations and modifications may be made while remaining within the spirit and scope of the invention.

1. An automated method for identifying the location of and disrupting an extracellular matrix structure, the method comprising:
using a substantially automated process to identify an X,Y predetermined position within the extracellular matrix structure on a surface of a culture plate; and
using a substantially automated process to disrupt the extracellular matrix structure at the X,Y predetermined position of the extracellular matrix structure on the surface of the culture plate with a pipette tip associated with a liquid handler.

2. The method according to claim 1, further comprising, prior to disrupting, identifying the location of the extracellular matrix structure on the surface of the culture plate with a substantially automated process.

3. The method according to claim 1, wherein:
the extracellular matrix structure is a hydrogel; and/or
the X,Y predetermined position is at or near the center of the extracellular matrix structure; and/or
the X,Y predetermined position of the extracellular matrix structure is determined by one or more parameters selected from the group consisting of the original seeding position on the surface of the culture plate and imaging of the extracellular matrix structure on the surface of the culture plate; and/or
a diameter of the extracellular matrix structure is determined by a volume of the extracellular matrix deposited on the surface of the culture plate or imaging of the extracellular matrix structure on the surface of a culture plate.

4-6. (canceled)

7. The method according to claim 1, further comprising:
moving the pipette tip in a multiplicity of steps to free the cells from the extracellular matrix structure, each of the multiplicity of moving steps comprising movement to an additional X,Y predetermined position within the extracellular matrix structure;
wherein the surface of the culture plate is a culture cell well.

8. (canceled)

9. The method according to claim 7, wherein each of the X,Y predetermined positions and the additional X,Y predetermined positions within the cell culture well are calculated according to the following:
$$X ( stepnumber ) = Xwellcenter + alpha / steps * stepnumber * COS ( theta / steps * stepnumber ) ;$$
$$and$$
$$Y ( stepnumber ) = Ywellcenter + alpha / steps * stepnumber * SIN ( theta / steps * stepnumber ) ,$$
wherein:
alpha=extracellular matrix structure diameter (mm)/1.8;
steps=number of steps for moving the pipette tip through the extracellular matrix structure, optionally an integer from 2 to 20;
Xwellcenter=x position of dome center within the well;
Ywellcenter=y position of dome center within the well; and
theta=a constant value between 5 and 50.

10. The method according to claim 7, wherein the X,Y predetermined positions and the additional X,Y positions form a pattern selected from the group consisting of a spiral pattern, a star pattern, and a zig-zag pattern within and/or through the extracellular matrix structure.

11. (canceled)

12. The method according to claim 7, wherein the liquid handler:
aspirates the pipette tip, and/or
dispenses a liquid medium from the pipette tip
within the extracellular matrix structure at one or more, two or more, a multiplicity of, or each of the X,Y predetermined positions.

13. The method according to claim 12, wherein aspirating and/or dispensing are each repeated one or more, two or more, a multiplicity of times at each X,Y predetermined positions.

14. The method according claim 12, wherein the aspirating or dispensing occurs at a fixed height between about 1 mm and about 3 mm above the bottom of the culture plate and at a fixed speed between about 1 μL/s and about 600 μL/s.

15-16. (canceled)

17. The method according to claim 14, wherein a cell dissociation reagent is added to the extracellular matrix structure at a fixed height between about 0.05 mm and about 0.3 mm above the culture plate surface.

18. (canceled)

19. The method according to claim 1, the method further comprising:
centrifuging a cell solution in a container to form a cell pellet and a supernatant;
aspirating a first volume of the supernatant at a first fixed height from the bottom of the container and a first fixed speed; and
aspirating a second volume of the supernatant at a second fixed height from the bottom of the container and a second fixed speed.

20. The method according to claim 19, wherein:
the cell pellet comprises organoids; and/or
the first volume is between about 400 uL and about 600 uL; and/or
first fixed height is between about 20 mm and about 40 mm from the bottom of the container; and/or
the first fixed speed is between about 200 μL/s and about 300 μL/s; and/or
wherein the second volume is between about 300 μL and about 500 μL; and/or
the second fixed height is between about 2 mm and about 8 mm from the bottom of the container; and/or
the second fixed speed is between about 35 μL/s and about 65 μL/s.

21-26. (canceled)

27. The method according to claim 1, the method further comprising:
providing a container comprising an organoid pellet and a supernatant of a first volume,
a) aspirating and dispensing a second volume at a first fixed height from the bottom of the container multiple times sequentially;
b) aspirating a third volume at a second fixed height from the bottom of the container and dispensing the third volume back into the container; and
c) repeating the steps a) and b).

28. (canceled)

29. The method according to claim 27, wherein:
the first volume is between about 75 uL and about 125 uL; and/or
the second volume is between about 70 uL and about 90 uL; and/or
the first height is between about 0.1 mm and about 0.3 mm; and/or
the third volume is between about 75 uL and about 125 uL; and/or
the second height is between about 0.1 mm and about 0.3 mm.

30-33. (canceled)

34. The method according to claim 27, wherein step a) comprises aspirating and dispensing the second volume between about 40 times and about 60 times.

35. The method according to claim 1, the method further comprising:
a) mixing, via aspirating and dispensing, about 60% of the volume of a solution of cells multiple times;
b) aspirating a portion of the mixed solution to provide an aspirated solution and remaining non-aspirated solution;
c) dispensing at least a portion of the aspirated solution to a culture plate not previously used to culture the cells; and
d) repeating steps a) through c) with the remaining non-aspirated solution.

36. The method according to claim 35, wherein:
the solution of cells comprises organoids or fragments thereof; or
the solution of cells comprises an extracellular matrix.

37. (canceled)

38. The method according to claim 35, wherein the mixing via aspirating and dispensing is conducted at a fixed height of between about 2 mm and about 4 mm from the bottom of a container in which the solution of cells is located.

39. The method according to claim 35, wherein when conducting step d):
aspirating and dispensing is conducted at a fixed height from the bottom of a container in which the solution of cells is located that is smaller than a previous round of steps a) and b); or
aspirating and dispensing is conducted at a fixed height from the bottom of a container in which the solution of cells is located that is about ⅓ the height of a first round of steps a) and b).

40. (canceled)

41. The method according to claim 35, wherein dispensing at least a portion of the aspirated solution to the second culture plate comprises dispensing the aspirated solution at a distance of between about 0.1 mm and 0.5 mm from the surface of the culture plate not previously used to culture the cells.
